(19) United States
(12) Patent Application Publication
(10) Pub. No.: US 2008/0147152 A1
(43) Pub. Date: Jun. 19, 2008

(54) Self-activated warming device

(75) Inventors: Roger B. Quincy, Cumming, GA (US); Robert D. Wright, Peachtree City, GA (US)

Correspondence Address: DORITY & MANNING, P.A., POST OFFICE BOX 1449, GREENVILLE, SC, 29602-1449, US

(73) Assignee: KIMBERLY-CLARK WORLDWIDE, INC.

(21) Appl. No.: 11/639,967

(22) Filed: Dec. 15, 2006

(51) Int. Cl. A61F 7/03

(52) U.S. Cl. 607/112; 607/114

(57) ABSTRACT

A warming device for placement against a body part or object includes a shell member having an outer face and an inner face disposed against a surface to be warmed. A first warming substrate is disposed within the shell member and is an absorbent web having a generally uniform application of a first warming composition applied thereto, the warming composition activated by contact with an aqueous liquid. An aqueous liquid source is disposed within the shell member and is separated from the first warming substrate by a barrier member. The device is activated by manual manipulation to breach the barrier member causing liquid from the liquid source to move within the interior space of the shell member to contact and activate the warming composition whereby a warming reaction is generated.

### BACKGROUND OF THE INVENTION

Thermal heat wraps or packs are well known in the art for providing any manner of therapeutic or soothing warming to a person. For example, such devices are typically used to treat sports injuries, or used in various medical procedures to warm a patient. Warming packs are also used extensively in various “cold” recreational activities. For example, hunters or skiers may place such a device in their gloves or boots to keep the extremities warm in extreme cold conditions.

WO 2004/105709 describes various types of self-warming skin care compositions that include a heat generating powder coated with a combination of oils and/or waxes to minimize a later reaction rate when the powder is combined with water, and to enable an increased duration heating cycle. The heat generated in the exothermic reaction of the powder with water warms the skin, enhances cleansing, and may provide improved blood circulation. The heat generating powder may be an inorganic salt, such as calcium chloride, magnesium chloride, magnesium sulphate, and the like.

U.S. Pat. Application 2005/0136765 describes various fibrous sheet materials, such as tissue and paper towels, provided with chemical agents, such as certain salts, that create a temperature change in the sheet when exposed to water. When water is absorbed by the sheet, the salt dissolves and produces a cooling or warming temperature change depending on the particular salt. The sheets may be used for wiping the hands, face, and body since the temperature change can be initiated by absorption of body fluids, such as sweat or surface moisture on the skin.

Unfortunately, various problems arise when attempting to apply exothermic compositions to a substrate. Specifically, if the exothermic composition were exposed to moisture during application or storage, the exothermic reaction could occur prematurely. Certain salts are susceptible to absorbing moisture from the air and may slowly warm over time until an equilibrium moisture rate is reached. This ultimately would lower the quality of the exothermic composition and degrade the desired effect of any device incorporating the composition. Also, it is difficult to apply the exothermic compositions to substrates in an effective and uniform concentration that yields the desired result.

Various techniques have been suggested in an attempt to overcome certain inherent problems with thermal devices that incorporate an exothermic composition. For example, U.S. Pat. No. 6,436,128 to Usui describes an exothermic composition that contains an exothermic substance, a water-absorptive polymer and/or tackifier, a carbon component and/or metal halide, and water. An excessive amount of water is used in the composition to suppress a premature oxidation reaction with air. Once formulated, the exothermic composition of Usui is laminated and sealed in a thin pouch. The pouch absorbs water from the composition so that, when the seal is broken, the exothermic reaction may proceed upon exposure to air and moisture. Despite overcoming certain problems of conventional techniques, Usui is still too complex for many consumer applications. Moreover, it is often difficult to control the reaction rate of the exothermic substance in such devices.

As such, the art is continuously seeking improved thermal warming devices that are simple, effective, relatively inexpensive to make, and also readily controllable.

### SUMMARY OF THE INVENTION

Objects and advantages of the invention will be set forth in part in the following description, or may be obvious from the description, or may be learned through practice of the invention.

In accordance with one embodiment of the present invention, a self-activated thermal warming device is provided. The device is not limited by its intended use, and may find utility in the medical arts as a patient warming or therapeutic device, or in the recreational arts as a personal warmer to be placed in an article such as a glove, shoe, boot, and so forth. The device may also have utility in the food industry as a device to maintain the temperature of heated food products. In particular embodiments, the device is a disposable single-use wrap or sleeve configured to be removably applied to any part of a person's body or other object to supply warmth thereto.

The device includes a shell member having dimensions suited for the desired use of the device. For example, the device may have dimensions similar to conventional heating pads for being placed on a person's back, shoulder, and so forth. The shell may be configured as a wrap to be placed around a person's arm or leg. The shell includes an outer face and an inner face that is disposed against the surface to be warmed in use of the device. A first warming substrate is disposed within an interior space in the shell member. The warming substrate includes a material web having a generally uniform application of a first warming composition applied thereto, the warming composition being activated by contact with an aqueous liquid. This web may be a hydrophobic material.

A source of aqueous liquid is disposed within the interior space of the shell and is separated from the first warming substrate by a barrier member, such as a breakable wall, frangible seal, bladder or other breakable liquid container inserted into the shell member, or other suitable liquid source that is maintained separate from the warming substrate. The device may be activated by simple manual manipulation of the shell member to break, rupture, or otherwise breach the barrier member to release the liquid from the liquid source. As the liquid moves through the interior space and contacts the warming substrate, an exothermic warming reaction is generated. The manual manipulation may include, for example, any combination of twisting, pulling, compressing, squeezing, or bunching of the shell member.

In one embodiment, the shell member may define a generally rigid configuration, such as an open or closed-end cylinder, that maintains its dimensions when not in use. For example, the shell member may include a layer of foam insulation material, or other material of sufficient thickness and rigidity such that the device maintains its cylindrical configuration without an article or body part being inserted therein.

In an alternate embodiment, the shell member is defined by a flexible sleeve member that is readily conformable around an object or body part, and assumes a generally flat or folded configuration when not in use. In this embodiment, the shell member may be formed by any combination of flexible and conformable materials. The first warming substrate is also formed from a flexible material so as to conform with the flexible sleeve. The flexible sleeve may be a planar component with any suitable attaching mechanism at one or both of the ends thereof so that the sleeve can be wrapped around and attached to an article or body part. In still another embodiment, the flexible sleeve may be formed into a continuous loop that is opened by the user to insert an arm, leg, or object into the device.

The warming substrate may be a separate web material that is placed into the interior space of the shell member, and may take on any size, shape, number, and configuration within the interior space of the shell member. For example, the substrate may have dimensions essentially matching the interior length and width dimensions of the shell member. Multiple substrates may be provided and separated by a material layer that serves to conduct fluid between the substrates. Alternatively, the warming substrate may be defined by an interior layer of the shell member. For example, the shell member may be a laminate material having a nonwoven material layer exposed within the interior of the shell. This nonwoven layer may have the warming composition applied thereto and also function as the warming substrate.

In a particular embodiment, the warming substrate may be a dual layer bonded carded web material, as described herein.

The device may include one or a combination of different warming substrates having the same or different warming compositions. For example, the first warming composition may be applied to a first substrate to provide the device with a first set of warming characteristics. A second warming composition may be applied to the same substrate, or a second substrate, to provide a different set of warming characteristics. Alternatively, the same warming composition may be applied in different concentration levels to the same or different substrates to provide different sets of warming characteristics. In a particular embodiment, a first set of warming characteristics may be desired to deliver an initial rapid and pronounced increase in temperature, while a second set of warming characteristics may provide a more gradual and sustained temperature increase. Any combination of warming compositions may be utilized in this regard to achieve any desired warming profile.

The warming compositions may vary within the scope and spirit of the invention. In one embodiment, the warming composition is an aqueous solution coated onto the substrate, wherein the warming agent re-crystallizes on the substrate upon drying the solution. The solution may include any combination of binder, viscosity modifier, or other component to aid in application of the warming composition to the substrate.

The liquid source within the interior space of the shell may take on various configurations. For instance, the source may be defined by a liquid-filled compartment defined within the shell, the compartment having a barrier wall or seal that is breached or opened by simple manual manipulation of the device. This compartment may be defined by one or more of the interior surfaces of the shell member. For example, a frangible wall may be attached at the longitudinal ends of the shell member between the opposite interior surfaces of the shell member, with this wall rupturing or breaking upon pressure being applied to the device. In another embodiment, the compartment may be defined entirely by the interior surfaces of the shell member, with a frangible seal between the surfaces separating the liquid compartment from the warming substrate. In other embodiments, the liquid source includes any combination of separate liquid filled “bladders” or breakable containers placed within the interior of the shell. These bladders may include, for example, vials, pliable pouches, or any other suitable liquid container that is readily opened or breeched by external manual manipulation of the device.

Desirably, the amount of liquid released into the interior space of the shell member is calculated such that essentially all of the liquid is absorbed by the substrate and warming composition. In this manner, excess liquid is not held within said interior space after activation of said device.

Other features and aspects of the present invention are described in more detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1A is a perspective and partial cut-away view of an embodiment of a warming device in accordance with the invention.

FIG. 1B is a perspective view of the embodiment of FIG. 1A wrapped around a body part of a person.

FIG. 1C is a cross-sectional view taken along the lines indicated in FIG. 1A.

FIG. 2 is a perspective view of an alternative embodiment of a warming device according to the invention in the form of a flexible closed-loop device.

FIG. 3 is a perspective view of still another embodiment of a molded warming device for a beverage container in accordance with the invention.

FIG. 4 is a cross-sectional view of a particular embodiment of a warming device in accordance with the invention.

FIG. 5 is a cross-sectional view of an alternative embodiment of a warming device in accordance with the invention.

FIG. 6 is a cross-sectional view of still a different embodiment of a warming device in accordance with the invention.

FIG. 7 is a partial cross-sectional view particularly illustrating an embodiment of a panel member that may be used as a shell member component in accordance with the invention.

FIGS. 8A and 8B are time v. temperature charts for Examples 1 and 2, respectively.

### DETAILED DESCRIPTION OF REPRESENTATIVE EMBODIMENTS

Reference now will be made in detail to various embodiments of the invention, one or more examples of which are set forth below. Each example is provided by way of explanation, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention cover such modifications and variations.

Generally speaking, the present invention is directed to a warming device that is particularly suited as a warming sleeve or wrap to be applied against any desired body part or object. The device includes a warming composition that generates a warming effect upon activation of the device. Through selective control of the warming composition and supply of aqueous reactant, a desired warming profile may be achieved in which an increased temperature is reached quickly and maintained over an extended period of time. For example, an increased temperature of from about 5° C. to about 15° C. may be achieved in about 3 minutes or less and sustained at an increased temperature for a time sufficient for the device to provide a therapeutic warming effect.

The warming composition may include any one or combination of known warming agents that react with an aqueous liquid in an exothermic physical reaction that produces heat by the positive heat of dissolution of the agent into the aqueous liquid. For example, the dissolution in water of certain inorganic salts, such as magnesium chloride, produce heat. Examples of other useful inorganic salts include calcium chloride, aluminum chloride, ferric chloride, and zinc chloride; sulphates such as magnesium sulphate, zinc sulphate, ferrous sulphate, aluminum sulphate, calcium sulphate, and sodium sulphate; and the like. A preferred warming agent is magnesium chloride, commercially available in powder form, or in the form of a low or high-density prill. The low-density prills may also include a clay binder, such as kaolin, at a low percentage by weight (from about 0.5 to about 5% by weight, often from about 1 to 3% by weight).

In a particular embodiment, the warming composition is applied in solution form to the warming substrate, wherein the warming agent permeates through the porous substrate and recrystallizes on the substrate upon drying the solution.

The solution may also employ a binder for enhancing the durability of the composition when applied to the warming substrate. The binder may also serve as an adhesive for bonding one substrate to another substrate. Generally speaking, any of a variety of binders may be used in the warming composition of the present invention. Suitable binders may include, for instance, those that become insoluble in water upon crosslinking. Crosslinking may be achieved in a variety of ways, including by reaction of the binder with a polyfunctional crosslinking agent. Examples of such crosslinking agents include, but are not limited to, dimethylol urea melamine-formaldehyde, urea-formaldehyde, polyamide epichlorohydrin, etc. In some embodiments, a polymer latex may be employed as the binder.

Water-soluble organic polymers may also be employed as binders, either alone or in conjunction with the polymer latexes. For example, one class of suitable water-soluble organic polymers is polysaccharides and derivatives thereof. Polysaccharides are polymers containing repeated carbohydrate units, which may be cationic, anionic, nonionic, and/or amphoteric. In one particular embodiment, the polysaccharide is a nonionic, cationic, anionic, and/or amphoteric cellulosic ether. Suitable nonionic cellulosic ethers may include, but are not limited to, alkyl cellulose ethers, such as methyl cellulose and ethyl cellulose; hydroxyalkyl cellulose ethers, such as hydroxyethyl cellulose, hydroxypropyl cellulose, hydroxypropyl hydroxybutyl cellulose, hydroxyethyl hydroxypropyl cellulose, hydroxyethyl hydroxybutyl cellulose and hydroxyethyl hydroxypropyl hydroxybutyl cellulose; alkyl hydroxyalkyl cellulose ethers, such as methyl hydroxyethyl cellulose, methyl hydroxypropyl cellulose, ethyl hydroxyethyl cellulose, ethyl hydroxypropyl cellulose, methyl ethyl hydroxyethyl cellulose and methyl ethyl hydroxypropyl cellulose; and so forth. Suitable examples of gel-forming anionic polysaccharides include natural gums, such as gellan gum and alginate gums (e.g., ammonium and alkali metal of salts of alginic acid); chitosan; carboxymethylcellulose, pectins, carrageenan, xantham gum, and derivatives or salts thereof.

The concentration of the binder in the warming composition will generally vary based on the desired properties of the warming substrate. For example, although relatively high binder concentrations may provide better physical properties for the warming composition, they may likewise have an adverse effect on other properties, such as the absorptive capacity of the substrate to which it is applied. Conversely, relatively low binder concentrations may reduce the ability of the warming agent component of the composition to remain affixed on the substrate. Thus, in most embodiments, the binder is present in the warming composition solution in an amount from about 0.01 wt. % to about 20 wt. %, in some embodiments from about 0.1 wt. % to about 10 wt. %, and in some embodiments, from about 0.5 wt. % to about 5 wt. %.

Viscosity modifiers may be used in the warming composition, for example, to adjust the viscosity of the coating formulation based on the desired coating process and/or performance of the coated warming substrate. Suitable viscosity modifiers may include gums, such as xanthan gum, which also functions as a binder. Other binders, such as the cellulosic ethers, may also function as suitable viscosity modifiers. When employed, such additional components typically constitute less than about 5 wt. %, in some embodiments less than about 2 wt. %, and in some embodiments, from about 0.001 wt. % to about 1 wt. % of the coating formulation.

A polymer emulsion barrier coating, such as Airflex® EP1188 from Air Products and Chemicals, Inc., may also be added to the solution in an amount to render the substrate material more hydrophobic. Any one or combination of commercially available barrier coating compositions may be used for this purpose.

Any type of porous substrate may be coated with the warming composition in accordance with the present invention. For instance, nonwoven fabrics, woven fabrics, knit fabrics, paper web, film, absorbent foams, etc., may be applied with the warming composition. When utilized, nonwoven fabrics may include, but are not limited to, spunbonded webs (apertured or non-apertured), meltblown webs, bonded carded webs, air-laid webs, coform webs, hydraulically entangled webs, and so forth.

In certain embodiments, the substrate is an absorbent material that captures the liquid from the liquid source to generate a uniform warming effect in the device regardless of orientation or position of the shell member. The absorbent material may include an absorbent web formed using any technique, such as a dry-forming technique, an airlaying technique, a carding technique, a meltblown or spunbond technique, a wet-forming technique, a foam-forming technique, etc. The absorbent layer may contain cellulosic fibers, such as natural and/or synthetic fluff pulp fibers. The fluff pulp fibers may be kraft pulp, sulfite pulp, thermomechanical pulp, etc. In addition, the fluff pulp fibers may include high-average fiber length pulp, low-average fiber length pulp, or mixtures of the same. One example of suitable high-average length fluff pulp fibers includes softwood kraft pulp fibers.

In a particular embodiment, the substrate is a dual layer bonded carded web material of type used, for example, as surge layer materials in absorbent articles. Examples of these dual layer bonded carded webs may be found in U.S. Pat. No. 5,820,973 Heterogeneous Surge Material for Absorbent Articles, incorporated herein by reference for all purposes.

To apply the warming composition to the substrate, the components may initially be dissolved or dispersed in a solvent. For example, one or more of the above-mentioned components may be mixed with a solvent, either sequentially or simultaneously, to form a coating formulation that may be easily applied to a substrate. Any solvent capable of dispersing or dissolving the components is suitable, for example water; alcohols such as ethanol or methanol; dimethylformamide; dimethyl sulfoxide; hydrocarbons such as pentane, butane, heptane, hexane, toluene and xylene; ethers such as diethyl ether and tetrahydrofuran; ketones and aldehydes such as acetone and methyl ethyl ketone; acids such as acetic acid and formic acid; and halogenated solvents such as dichloromethane and carbon tetrachloride; as well as mixtures thereof. In one particular embodiment, for example, water is used as the solvent so that an aqueous coating formulation is formed. Although the actual concentration of solvent (e.g., water) employed will generally depend on the type and amount of warming agent and the substrate on which it is applied, it is nonetheless typically present in an amount from about 10 wt. % to about 80 wt. % of the coating formulation. The amount of the other components added to the coating formulation may vary depending on the amount of warmth desired, the wet pick-up of the application method utilized, etc.

The viscosity of the warming composition formulation may be varied in accordance with the coating method to achieve the desired heating profile as a function of the amount of the composition on the substrate. For instance, lower viscosities may be employed for saturation coating techniques (e.g., dip-coating), while higher viscosities may be employed for drop-coating techniques. If desired, thickeners or other viscosity modifiers may be employed in the coating formulation to increase or decrease viscosity. The coating formulation may have a solids content of from about 10% to about 80%, in some embodiments from about 30% to about 70%, and in some embodiments, from about 50% to about 60%. By varying the solids content of the coating formulation, the presence of the warming agent powder and other components in the exothermic composition may be controlled. For example, to form an exothermic composition with a higher level of warming agent, the coating formulation may be provided with a relatively high solids content so that a greater percentage of the agent is incorporated into the exothermic composition during the application process. If desired, thickeners or other viscosity modifiers may be employed in the coating formulation to increase or decrease viscosity.

The warming composition formulation may be applied to a substrate using any conventional technique, such as bar, roll, knife, curtain, print (e.g., rotogravure), spray, slot-die, drop-coating, or dip-coating techniques. The materials that form the substrate (e.g., fibers) may be coated before and/or after incorporation into the substrate. The composition may be applied to one or both surfaces of the substrate. For example, the composition may be present only on the surface of the substrate that is adjacent the inner face of the shell member, or on both surfaces of the substrate. In addition, the composition may cover an entire surface of the substrate, or may only cover a portion of the surface.

Regardless of the manner in which the warming composition is applied, the resulting warming substrate is typically heated to a certain temperature to remove the solvent and re-crystallize the warming agent. For example, the thermal substrate may be heated to a temperature of at least about 100° C., in some embodiments at least about 110° C., and in some embodiments, at least about 120° C. In this manner, the resulting dried warming composition is anhydrous, i.e., generally free of water, and is distributed throughout the substrate in the form of a film-like network of re-crystallized warming agent. By minimizing the amount of moisture, the composition is less likely to react prematurely and generate warmth. Thus, the composition may remain inactive for extended periods until it is desired to use the warming device.

The thickness of the warming composition may also vary. For example, the thickness may range from about 0.01 millimeters to about 5 millimeters, in some embodiments, from about 0.01 millimeters to about 3 millimeters, and in some embodiments, from about 0.1 millimeters to about 2 millimeters. In some cases, a relatively thin coating may be employed (e.g., from about 0.01 millimeters to about 0.5 millimeters). Such a thin coating may enhance the flexibility of the substrate.

To maintain absorbency, porosity, flexibility, and/or some other characteristic of the substrate, it may sometimes be desired to apply the warming composition so as to cover less than 100%, in some embodiments from about 10% to about 80%, and in some embodiments, from about 20% to about 60% of the area of one or more surfaces of the substrate. For instance, in one particular embodiment, the warming composition is applied to the substrate in a defined pattern (e.g., reticular pattern, diamond-shaped grid, dots, and so forth). Such a patterned composition may provide sufficient warming to the substrate without covering a substantial portion of the surface area of the substrate. This may be desired to optimize flexibility, absorbency, or other characteristics of the substrate. It should be understood, however, that the coating may also be applied uniformly to one or more surfaces of the substrate.

In addition, a patterned application of the warming composition may also provide different warming characteristics (functionality) to each zone. For example, in one embodiment, the substrate is treated with two or more patterns of coated regions that may or may not overlap. The regions may be on the same or different surfaces of the substrate. One region may be coated with a first warming composition, while another region is coated with a second different warming composition. The first region may provide a rapid but relatively short warming profile, while the second region generates a gradual but sustained warming profile. In embodiments wherein different warming compositions are applied to the same substrate, care must be taken to separate the compositions during application and drying. In alternate embodiments, the different warming compositions may be applied to different substrates.

Other substrates may also be employed to improve or enhance application of warming through the shell member to an article or body part. For example, any number or combination of thermal conductive and/or insulation material layers may be disposed within the interior space of the shell member. These materials may be employed to provide warming to substantially only the inner face of the shell member that is placed against an article or body part. In an alternative arrangement, the shell member is reversible such that either face may provide the desired warming effect, with the materials disposed within the shell member to provide warming to both faces of the shell member.

For example, the thermal device may employ a thermally conductive layer to help distribute the generated heat along the x-y plane of the device, thereby improving the uniformity of warmth application. Although any thermally conductive material may generally be employed, it is often desired that the selected material be flexible and conformable. Suitable conformable materials include, for instance, fibrous materials (e.g., nonwoven webs), films, and so forth. For example, the thermally conductive layer may contain a nonwoven laminate, such as a spunbond/meltblown/spunbond (“SMS”) laminate. The SMS laminate is formed by well-known methods, such as described in U.S. Pat. No. 5,213,881 to Timmons, et al., which is incorporated herein its entirety by reference thereto for all purposes. A variety of techniques may be employed to provide conductivity to the thermally conductive layer. For example, a metallic coating may be utilized to provide conductivity. Metals suitable for such a purpose include, but are not limited to, copper, silver, nickel, zinc, tin, palladium, lead, aluminum, molybdenum, titanium, iron, and so forth. Metallic coatings may be formed on a material using any of a variety of known techniques, such as vacuum evaporation, electrolytic plating, etc. For instance, U.S. Pat. No. 5,656,355 to Cohen; U.S. Pat. No. 5,599,585 to Cohen; U.S. Pat. No. 5,562,994 to Abba. et al.; and U.S. Pat. No. 5,316,837 to Cohen, which are incorporated herein their entirety by reference thereto for all purposes, describes suitable techniques for depositing a metal coating onto a material.

Besides a metal coating, still other techniques may be employed to provide conductivity. For example, an additive may be incorporated into the material (e.g., fibers, film, etc.) to enhance conductivity. Examples of such additives include, but are not limited to, carbon fillers, such as carbon fibers and powders; metallic fillers, such as copper powder, steel, aluminum powder, and aluminum flakes; and ceramic fillers, such as boron nitride, aluminum nitride, and aluminum oxide. Commercially available examples of suitable conductive materials include, for instance, thermally conductive compounds available from LNP Engineering Plastics, Inc. of Exton, Pa. under the name Konduit® or from Cool Polymers of Warwick, R.I. under the name CoolPoly®. Although several examples of conductive materials have been described above, it should be understood that any known thermally conductive material may be generally used in the present invention.

As mentioned, an insulation layer may be employed to inhibit loss of heat to the outer environment. The insulation layer may be within the interior of the shell member, or attached to the exterior of the outer face of the shell member. Any known insulation material may be employed in this regard. If desired, the selected insulation material may be fibrous in nature to improve the overall conformability of the thermal device. The fibrous material may possess high loft to enhance its insulative properties. Suitable high loft materials may include porous woven materials, porous nonwoven materials, etc. Particularly suitable high loft materials are nonwoven multicomponent (e.g., bicomponent) polymeric webs. For example, the multicomponent polymers of such webs may be mechanically or chemically crimped to increase loft. Examples of suitable high loft materials are described in more detail in U.S. Pat. No. 5,382,400 to Pike, et al.; U.S. Pat. No. 5,418,945 to Pike, et al. and U.S. Pat. No. 5,906,879 to Huntoon, et al., which are incorporated herein in their entirety by reference thereto for all purposes. Still other suitable materials for use as an insulation material are described in U.S. Pat. No. 6,197,045 to Carson, which is incorporated herein in its entirety by reference thereto for all purposes.

Various foam materials may be utilized as the insulating foam layer in sleeves according to the invention. A particularly well-suited foam is a styrene based, low-density, open-cell foam made with balanced amounts of one or more surfactants and a plasticizing agent in a foam polymer formula. Thermoplastic elastomers can be added to the foam polymer formula to improve softness, flexibility, elasticity, and resiliency of the foam layer. The open-cell content of the foam is controlled by adjusting the amount of surfactant and/or plasticizing agent included in the foam polymer formulation, and in particular embodiments suited for the present invention, the open-cell content can be at about 80% or greater. This particular type of foam is described in detail in the published U.S. patent application Ser. No. 10/729,881 (Publication No, 20050124709) and U.S. patent application Ser. No. 11/218,825 (Publication No. 20060030632), both of which are incorporated herein for all purposes.

In addition, substrates may be employed to aid in distributing liquid from the liquid source throughout the interior of the shell member to the warming substrate to produce a more even warming effect. Such materials may include, for example, a nonwoven web, a film, a channeled or embossed substrate, and any other material that serves to wick or channel liquid from one area to another without absorbing or retaining the liquid to any significant degree.

The shell member is not limited to any particular shape or material. In particular embodiments, the shell member is liquid impervious and comprises a thin, flexible, envelope-type structure. The shell member is formed of materials that are not deleteriously affected by any of the contents of the warming composition, and which are resistant to the warm temperature produced by the device. The shell member may include a thermally conductive material at one or both interior surfaces. Such materials can be polymeric, and include ionomer film (for example, SURLYN available from DuPont), polyethylene, polypropylene, polyester (such as MYLAR film obtainable from DuPont) aluminum, aluminized polymer film, and other conventional plastic or other packaging materials suitable for containing warm liquids, such as rubber, vinyl, or vinyl-coated fabric. In a preferred embodiment, the thermally conductive material is a metal foil, such as one composed substantially of aluminum or copper, or a metallized plastic film such as aluminized polyester.

An insulation material layer may be provided at the outer face of the shell member to insulate the user from the warmth. This layer may also serve to present a soft, compliant, and functional surface to the user. This material may be, for example, a nonwoven material that is creped, embossed, textured, or otherwise presents a grip-enhanced surface to the user.

The shell member may be formed by a laminate material that includes a thermally conductive material laminated to an insulation layer material. For example, the shell member may be a laminate of a nonwoven insulation material and a thermally conductive film.

The shell member desirably has a thickness that permits the shell member to readily conform to the shape of the body part or object to be warmed. The shell member may be formed by separate material layers that are bonded together at the edges to form a hermetically sealed, substantially planar envelope. The edges of the material are bonded together by any suitable means, for example, soldering, heat sealing, ultrasonic welding, solvent welding, fold sealing, or the use of adhesives.

In still an alternate embodiment, the shell member may include a more rigid layer that defines a closed or open-ended configuration having dimensions to receive an article or body part (e.g., arm or foot) inserted therein. This layer may be, for example, an open or closed cell foam material. The interior space for receipt of the warming substrate may be defined within this material, or may be defined between the inner surface (surface that faces the container) of the foam and a thermally conductive layer that is attached to the interior surface.

The activating liquid is supplied by the internal liquid source. In a particular embodiment, this source is defined by a compartment within the shell member that is opened or breached by manual manipulation of the shell member by the user. The barrier may be a wall formed of a material that allows its rupture, break, perforate, or otherwise be compromised by manual deformation of the shell member, for example upon the user compressing or twisting the shell member prior to placing the device against an object or body part. Any number and configuration of barrier walls may be formed in the interior of the shell member depending on the size of the thermal device and the volume of liquid to be delivered. In one embodiment, the barrier comprises a brittle or weakened wall extending between the interior surfaces of the shell member. In another embodiment, the barrier may be a frangible seal between the opposing interior faces of the shell member.

In other embodiments, the liquid source includes any combination of separate liquid filled “bladders” placed within the interior of the shell. These bladders may include, for example, liquid vials, pliable pouches, or any other suitable liquid container that is readily opened, broken, or otherwise breeched by external manual manipulation of the device.

It may be desired that the shell member have elastic properties, particularly in the embodiments wherein the shell member defines a closed cylindrical sleeve. In this regard, the shell member may be formed of any combination of conventional liquid impermeable elastomeric materials, such as an elastomeric film/nonwoven laminate. The elastic component of the laminate can contain elastic strands or sections uniformly or randomly distributed throughout the material. Alternatively, the elastic component can be an elastic film or an elastic nonwoven web. In general, any material known in the art to possess elastomeric characteristics can be used in the present invention as an elastomeric component. Useful elastomeric materials can include, but are not limited to, films, foams, nonwoven materials, etc.

Other exemplary elastomeric materials which may be used include polyurethane elastomeric materials such as, for example, those available under the trademark ESTANE® from B.F. Goodrich & Co. or MORTHANE® from Morton Thiokol Corp., polyester elastomeric materials such as, for example, those available under the trade designation HYTREL® from E.I. DuPont De Nemours & Company, and those known as ARNITEL®, formerly available from Akzo Plastics of Amhem, Holland and now available from DSM of Sittard, Holland.

Another suitable material is a polyester block amide copolymer. Elastomeric polymers can also include copolymers of ethylene and at least one vinyl monomer such as, for example, vinyl acetates, unsaturated aliphatic monocarboxylic acids, and esters of such monocarboxylic acids. The elastomeric copolymers and formation of elastomeric nonwoven webs from those elastomeric copolymers are disclosed in, for example, U.S. Pat. No. 4,803,117.

When incorporating an elastomeric component, such as described above, into a base web, it is often desired that the elastomeric material form an elastic laminate with one or more other layers, such as foams, films, apertured films, and/or nonwoven webs. The elastic laminate generally contains layers that can be bonded together so that at least one of the layers has the characteristics of an elastic polymer. Examples of elastic laminates include, but are not limited to, stretch-bonded laminates and neck-bonded laminates.

The elastic member used in neck-bonded materials, stretch-bonded materials, stretch-bonded laminates, neck-bonded laminates and in other similar laminates can be made from materials, such as described above, that are formed into films, such as a microporous film, fibrous webs, such as a web made from meltblown fibers, spunbond filaments or foams. A film, for example, can be formed by extruding a filled elastomeric polymer and subsequently stretching it to render it microporous.

In one embodiment, the elastic member can be a neck stretched bonded laminate. As used herein, a neck stretched bonded laminate is defined as a laminate made from the combination of a neck-bonded laminate and a stretch-bonded laminate. Examples of necked stretched bonded laminates are disclosed in U.S. Pat. Nos. 5,114,781 and 5,116,662, which are both incorporated herein by reference. Of particular advantage, a necked stretch bonded laminate is stretchable in the machine direction and in a cross machine direction. Further, a neck stretch-bonded laminate can be made with a nonwoven basing that is texturized. In particular, the neck stretched bonded laminate can be made so as to include a nonwoven facing that gathers and becomes bunched so as to form a textured surface.

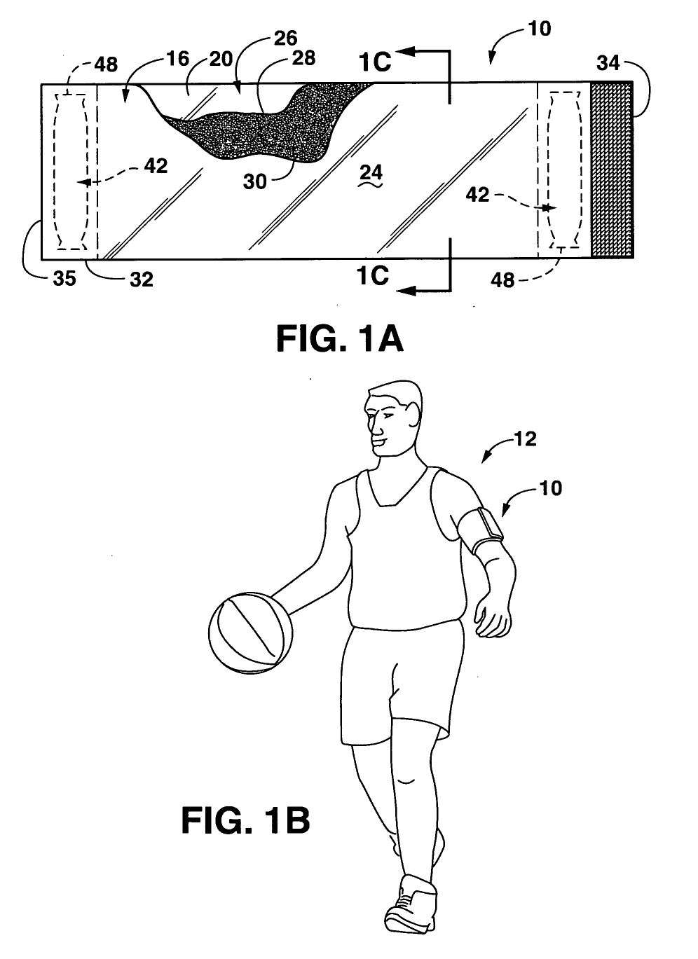

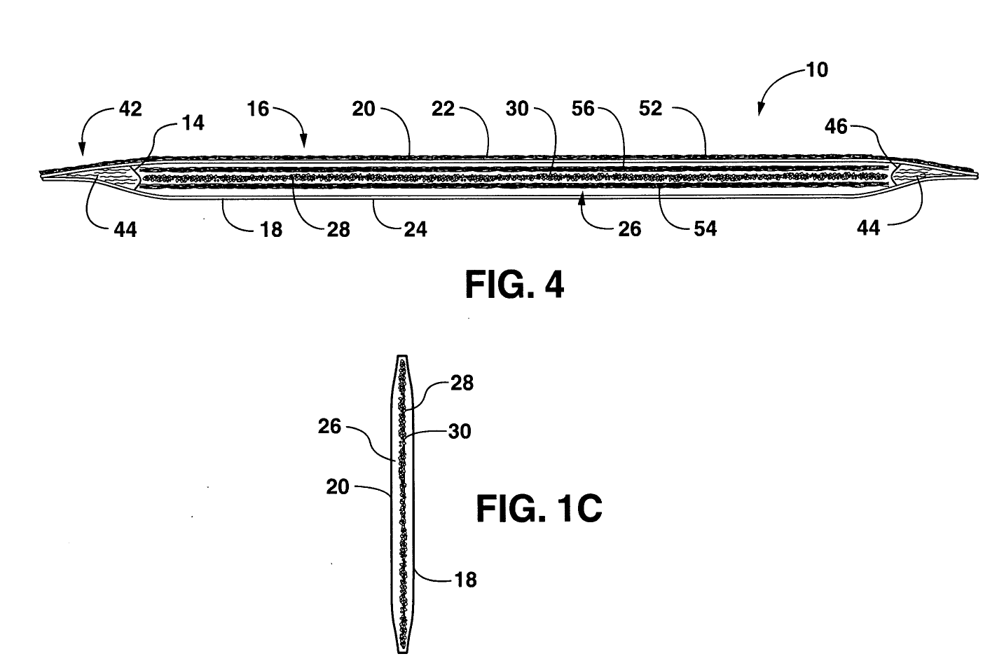

Various embodiments of a warming device 10 in accordance with the invention are illustrated in the figures. FIGS. 1A, 1B, and 1C illustrate a particular embodiment wherein the warming device 10 includes a shell member 16 formed from a first panel 18 and an opposite second panel 20 (FIG. 1C). These panel members may be the same material, or different materials. The first panel 18 defines an inner face 24 that is disposed against the surface to be warmed, such as a person's arm 12, when the sleeve 10 is wrapped around or otherwise applied against the surface, as illustrated in FIG. 1B. In the particular embodiment illustrated in FIGS. 1A through 1C, the shell member 16 defines a generally flat, planar, flexible sleeve member 32 having opposite ends 34, 35. At one of the ends, or at both ends, any suitable attaching mechanism is provided for securing the sleeve 32 around an object or body part 12. As particularly illustrated in FIGS. 1A and 1B, the attaching mechanism may be a conventional hook-and-loop type of fastener wherein hooks are provided along the end 34 of the sleeve 32. These hooks engage directly against the outer surface material of the panel 20, or a separate landing zone of hook compatible material may be provided on the panel 20. In alternative embodiments, the attaching mechanism may be a releasable adhesive, mechanical device, and so forth. It should be appreciated that the invention is not limited by any particular type of attaching mechanism for securing the flexible sleeve 32 around or to an object to be warmed.

Referring to FIG. 1A, the shell member 16 defines an interior space 26. A warming substrate 28 is contained within this interior space and includes a warming composition 30 applied thereto. The warming substrate 28 may comprise a base material with the warming composition 30 applied in solution form over essentially the entire surface thereof, as discussed in detail above.

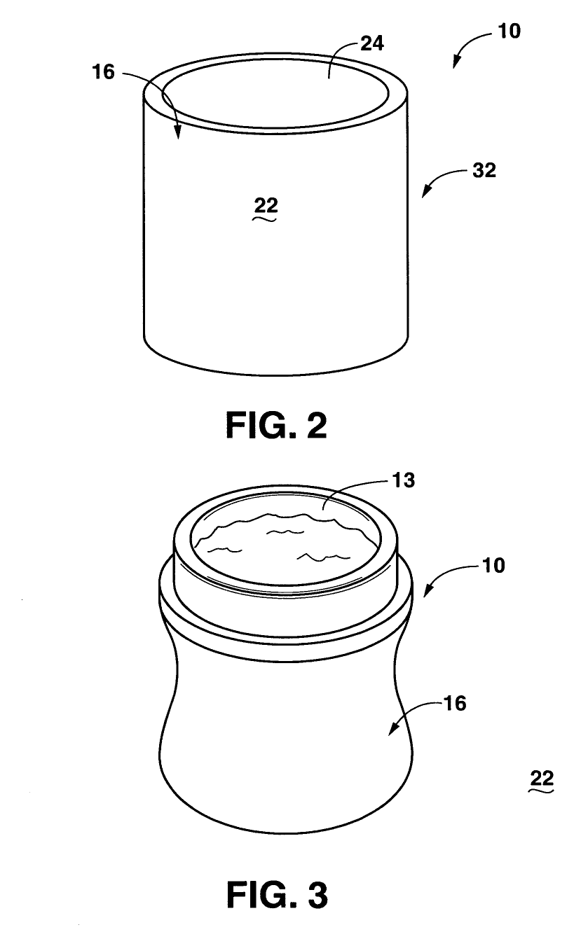

FIG. 2 illustrates an alternative embodiment of a warming device 10 wherein the shell member 16 is defined by a flexible sleeve member 32 that is formed into a closed-loop configuration. To use this device, the operator manipulates the sleeve member 32 into an open configuration, and subsequently slides an object or body part into the sleeve. This configuration may be particularly well suited as a therapeutic warming device to treat sports injuries.

FIG. 3 illustrates an alternative embodiment of a warming device wherein the shell member 16 is defined by a molded body having sufficient rigidity so as to maintain a generally cylindrical open or closed-ended receptacle configuration. This embodiment may be used to place an object, such as a beverage cup or container 13, into the warming device 10.

It should be appreciated that the warming device 10 according to the invention is not limited to any particular shape, configuration, or appearance. The unique thermal aspects of the present invention may be incorporated into any conventional style of warming or heating pad, food product insulator, and so forth.

Referring again to FIG. 1A, the warming substrate 28 desirably has a length and width dimension so as to completely encircle the object or body part 12 once the sleeve member 32 is applied around the item. In this regard, the substrate 28 may have dimensions corresponding to the width and length dimensions of the interior space 26. It should be appreciated, however, that the invention encompasses alternative embodiments wherein the substrate is discontinuous or does not completely encircle the item.

An aqueous liquid source 42 is disposed within the interior space 26 of the shell member 16. In the embodiment illustrated in FIG. 1A, the liquid source 42 is provided by bladders 48 disposed generally adjacent the opposite ends of the flexible sleeve 32, or at any other location within the interior space 26. The bladders 48 are inserted between the panel members 18, 20, in construction of the warming device 10. The bladders 48 are filled with an aqueous liquid, such as water, and rupture or burst upon sufficient pressure being applied thereto. To activate the device 10, a user simply grasps and squeezes the sleeve 32 at the ends thereof causing the bladders 48 to rupture and release the liquid contained therein. The liquid is then free to move within the interior space 26 and contact the warming composition 30 applied to the warming substrate 28. As discussed in detail above, the warming composition includes a warming agent that dissolves in the aqueous liquid and generates a warming effect. In a desirable embodiment, the base material of the warming substrate 28 is absorbent and captures the water released from the bladders 48. Desirably, the volume of liquid released from the bladders 48 is sufficient to saturate the absorbent web material of the warming substrate 28 to ensure a complete and effective warming reaction, while minimizing excess liquid that may tend to slosh around within the shell member 16.

In the embodiment of FIGS. 1A through 1C, it is understood that the barrier member between the warming substrate 28 and the liquid source 42 is the walls of the bladder 48 that rupture or otherwise break to release the liquid. Thus, in this embodiment, separate barrier walls or seals are not formed within the interior space 26 of the shell member 16.

FIG. 4 illustrates an embodiment of a warming device wherein the opposite panels 18 and 20 define the shell member 16 and interior space 26. In this particular embodiment, the liquid source 42 is provided by compartments 44 formed at the longitudinal ends of the device by an integral barrier 46, such as barrier walls 14 that extend between the inner surfaces of the panels 18, 20. These walls 14 may be formed by any material that breaks or ruptures upon external pressure being applied to the sleeve at the ends thereof to activate the device 10. The walls 14 may be thinned or weakened as compared to the panel members 18, 20 to ensure that they rupture or break prior to compromising the integrity of the panel members.

As discussed in detail above, the shell member 16, particularly the panels 18 and 20, may be formed of various suitable materials. In the embodiment illustrated in FIG. 4, the panel 18 defining the inner face 24 of the warming device 10 may be a liquid impermeable film. The opposite panel 20 may be a film/nonwoven laminate material wherein the nonwoven component of the laminate defines the outer face 22 that is presented to the user in use of the device. This nonwoven layer 22 presents a soft and compliant surface to the user, as compared to a film. The nonwoven layer may also serve as an insulation layer so that the user's hand is not exposed to the full warming effect of the device 10.

Still referring to FIG. 4, the warming substrate 28 with composition 30 applied thereto extends generally along the entire longitudinal length of the shell member 16 and is disposed between opposite material layers 56, 54. As discussed above, various material layers may be included within the interior space 26 to provide desirable thermal characteristics. For example, material layer 54 may be one of the thermally conductive materials described above. Material layer 56 may also be a conductive material, or a material specifically designed to quickly conduct the fluid released from the compartments 44 along the longitudinal length of the device. This material layer 56 may be, for example, a hydrophilic material having channels or other liquid conveying structure embossed or otherwise formed therein.

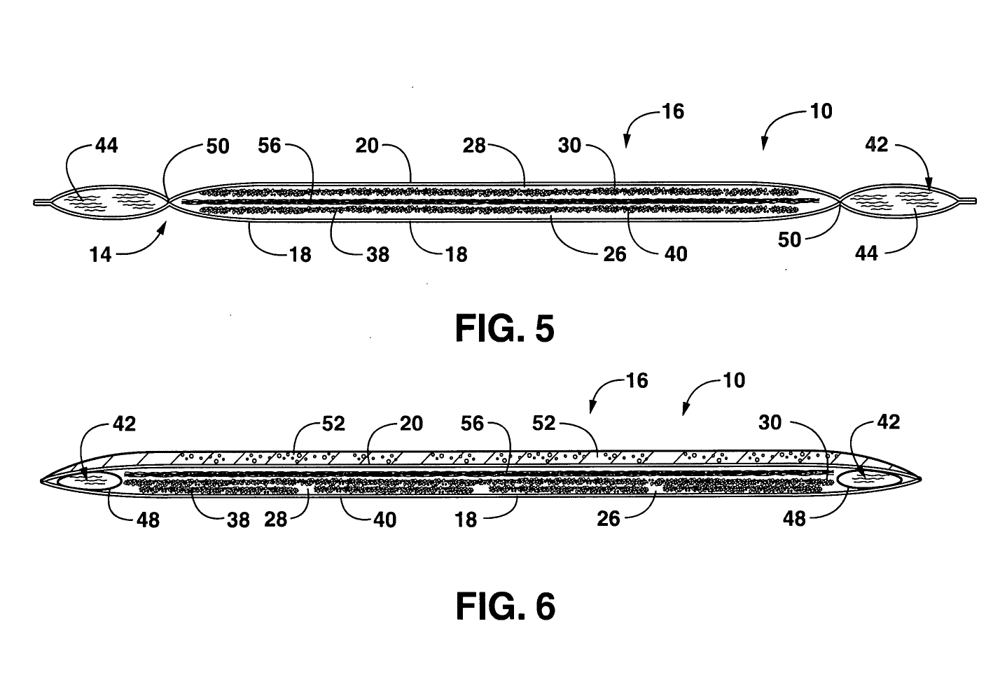

FIG. 5 illustrates an embodiment of a warming device wherein the liquid source 42 is defined by compartments 44 formed at the longitudinal ends of the shell member 16. The compartments 44 are formed by frangible seals 50 defined between the opposite panels 18, 20. These seals 50 may be formed by welding, adhesive, bonding, and the like, and have a seal strength that is less than the seals between the panel members 18 and 20 at the ends thereof to ensure the integrity of the sleeve member 16. To activate the device 10, a user applies external pressure to the compartment 44 causing the frangible seals 50 to separate and release the liquid contained within the compartments 44.

In the embodiment of FIG. 5, a second warming substrate 38 is provided with a second warming composition 40. The second substrate 38 and composition 40 may be identical to the first substrate and composition 28, 30, or may be completely different from the first combination. As discussed above, different combinations of warming substrates and compositions may be provided within any single warming device 10 to generate different warming profiles. In the embodiment of FIG. 5, a material layer 56 is provided between the substrates 28 and 38 and serves as a distribution layer to quickly channel the fluid from the compartments 44 along the longitudinal length of the substrates. The panel members 18 and 20 are formed, for example, of a thermally conductive and liquid impermeable film such that either surface may be applied against a body part or object. Thus, the embodiment of FIG. 5 is reversible and might include an appropriate attaching mechanism at one or both ends of the shell member 16.

FIG. 6 illustrates an embodiment of a warming device 10 wherein the panel 18 is defined by, for example, a liquid impermeable and thermally conductive film. The opposite panel 20 includes an interior film layer and an insulation layer 52 applied to the outer surface thereof. The insulation layer 52 may comprise, for example, a foam layer, and the entire panel member 20 may be a laminate of the film and foam material.

Still referring to the embodiment of FIG. 6, the liquid source 42 is defined by bladders 48 at the ends of the shell member 16. A first warming substrate 28 and composition 30 are provided within the interior space 26. A series of second warming substrates 38 and associate warming composition 40 are also provided. The second warming substrates 38 are discontinuous and the associated warming composition 40 may produce a substantially different set of warming characteristics as compared to the first warming substrate 28. A distribution material layer 56 may also be included within the interior space 26 to readily channel and distribute the liquid released from the bladders 48 along the longitudinal length of the shell member 16.

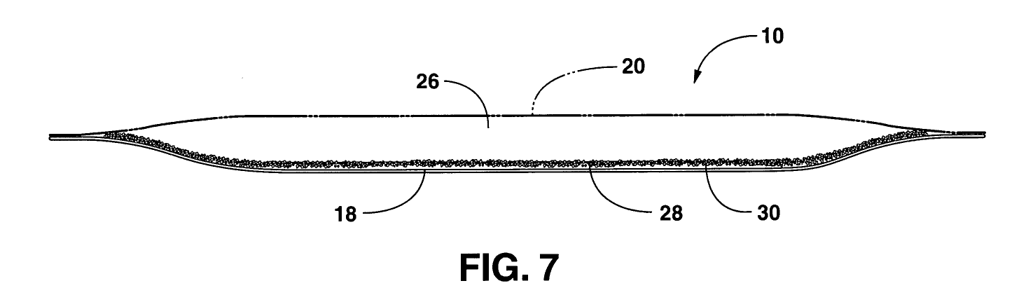

FIG. 7 illustrates a particular embodiment of a panel member 18 that incorporates the warming substrate 28 as an integral component thereof. In this particular embodiment, the panel member 18 may be a film/nonwoven laminate material, wherein the nonwoven component is coated with the warming composition 30. Thus, in this particular embodiment, the warming substrate 28 is not defined by a separate material layer that is disposed between opposite panel members.

The various layers and/or components of the warming device may be assembled together using any known attachment means, such as adhesives, ultrasonic bonding, thermal bonds, etc. Suitable adhesives may include, for example, hot melted adhesives, pressure-sensitive adhesives, and so forth.

The present invention may be better understood with reference to the following examples.

### EXAMPLE 1

The ability to form a water activated warming substrate was demonstrated. Initially, a dual layer bonded carded web was cut into pieces that measured 7 inches in the cross machine direction and 10 inches in the machine direction. One side of the web contained 17 gsm of a 100% 1.5 denier FiberVisions ESC 215 bicomponent (PE sheath/PP core) fiber with 0.55% HR6 finish. The other side of the web contained 58 gsm of a blend of 40% 6.0 denier Invista T-295 polyester fiber with 0.50% L1 finish and 60% of a 28 denier FiberVisions ESC 236 bicomponent (PE sheath/PP core) fiber with 0.55% HR6 finish. Therefore, the total basis weight of the dual layer bonded carded web was 75 gsm.

An aqueous coating formulation was prepared as follows. In a 400 milliliter PYREX® beaker, 255.5 grams of distilled water was heated to 70° C. Then, 5.3 grams of hydroxypropyl methylcellulose (Metolose 90SH-400, Shin-Etsu Chemical Co.) were added while stirring the warm water. Stirring was continued as the mixture was left to cool to room temperature. The viscosity of the hydroxypropyl methylcellulose (HPMC)/water solution was measured at about 420 centipoise using a Brookfield DV-1 viscometer with an LV-3 spindle set at 100 rpm. Next, 120.0 grams of the HPMC/water solution were placed in a 250 milliliter PYREX® beaker, and while stirring, 60.0 grams of magnesium chloride (Aldrich, −325 mesh, <5% water content) were slowly added. After the formulation was cooled to room temperature with a water bath, the viscosity was measured at about 3,970 centipoise using a Brookfield DV-1 viscometer with an LV-3 spindle set at 12 rpm. The calculated concentration of each component of the aqueous formulation is set forth below in Table 1.

TABLE 1
Components of the Aqueous Formulation

| Component | Calculated Amount |
|---|---|
| Magnesium Chloride | 33.3% |
| Hydroxpropyl Methylcellulose | 1.4% |
| Water | 65.3% |

The aqueous formulation was applied to the polyester/bicomponent fiber side of the dual layer bonded carded web using a #60 single wound coating rod. The coated fabric pieces were placed in a laboratory oven at 120° C. for about 30 minutes and then left overnight at 90° C. to complete the drying step. The concentration of the components of the coating composition was calculated from the coated and dried fabric pieces (29.8±0.4 grams), the untreated pieces of fabric (2.8±0.1 grams), and the composition of the aqueous formulation. The results are set forth below in Table 2.

TABLE 2
Components of the Coating Composition

| Component | Calculated Amount |
|---|---|
| Magnesium Chloride | 96.1% |
| Hydroxpropyl Methylcellulose | 3.9% |
| Solids Add-On Level | ~975% |

A sleeve structure (2.8″×10.5″) was then designed for activating the warming reaction. Specifically, the sleeve structure was produced with a material obtained from Ampac Flexibles. The material contained a film made with a 0.5 mil biaxial oriented nylon layer laminated adhesively to a 2.0 mil linear low density polyethylene layer which was laminated adhesively on the nylon side to a rib knit patterned spunbond nonwoven fabric (basis weight of 50 grams per square meter). The sleeve was constructed with the film layer on the inside and the fabric layer on the outside. The total basis weight of this film/fabric laminate was measured at 180 grams per square meter and the thickness was measured at 0.74 mm (0.029 in) using a Mitutoyo Digimatic Indicator. Four pieces (2″ CD×7″ MD) of the coated fabric (total weight of 25.4 grams) were placed inside of the sleeve. Two pieces were placed on each side of a 1.5 osy bonded carded web fabric (60% 1.5 denier FiberVisions ESC 215 bicomponent (PE sheath/PP core) fiber with 0.55% HR6 finish, 40% 6.0 denier Invista T-295 polyester fiber with 0.50% L1 finish) with the coated side of each piece aligned to face the sleeve. Two water bags were also placed inside of the film sleeve. The water bags were made out of GF-14 film (a 1.25 mil low density polyethylene barrier film from Pliant Corp.) and filled with 12.0 and 13.4 grams of water. The bags were constructed by folding over a 3.5-inch by 2.5-inch piece of the film and heat sealing two edges. The water was then added with a plastic syringe and the third edge was then heat sealed. The final dimensions of the bags were about 1.8 inches by 2.5 inches. A water bag was positioned at each end of the sleeve. The film/fabric laminate that formed the sleeve was also heat sealed to enclose the coated fabric pieces, the uncoated fabric piece, and the water bags.

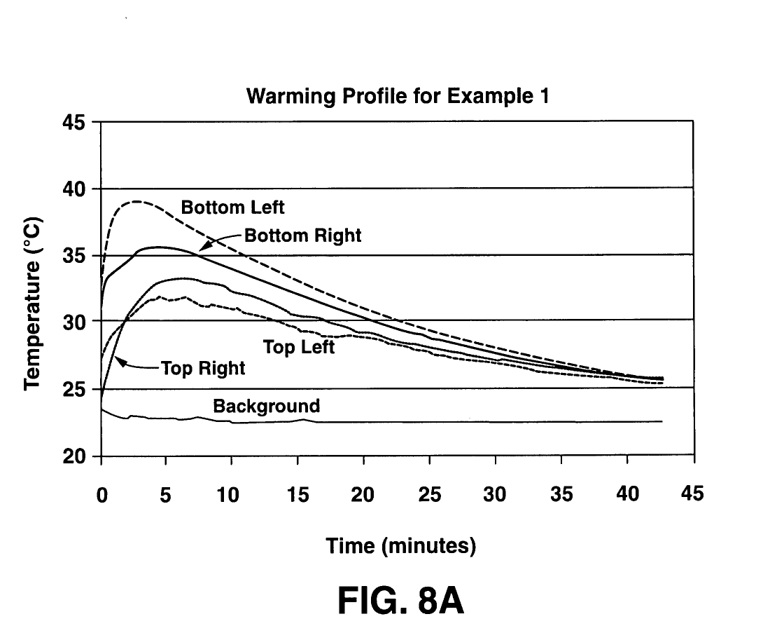

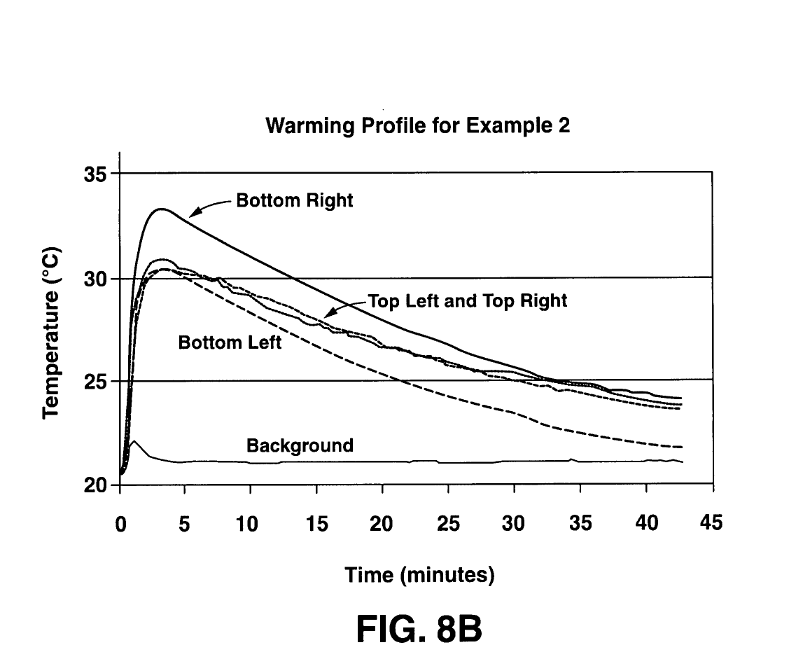

Four type K thermocouples (OMEGA Engineering, Incorporated) were attached to the sleeve to monitor the temperature as a function of time. The sleeve was placed on a layer of bubble wrap. A fifth type K thermocouple was attached to the bubble wrap to monitor the background temperature. Data from the thermocouples was collected with a Pico® TC-08 eight channel thermocouple data logger which was attached to a computer. The warming reaction was activated by squeezing the two water bags which broke at least one of the heat sealed edges and thus released the water. The data collection was started about 1 minute after breaking the water bags. FIG. 8A shows the warming data. Note that a warming effect was produced immediately after the fabrics inside of the sleeve were exposed to the water from the broken water bags.

### EXAMPLE 2

A second exothermic coating formulation was tested similar to the procedure described above with respect to Example 1.

The aqueous coating formulation included 168.0 grams of water mixed and stirred with 2.84 grams of xanthan gum (Verxan D from Cargill Corp.). Then, 85.4 grams of magnesium chloride (Aldrich, −325 mesh) were slowly added to the solution. After the solution had cooled to room temperature, the viscosity was measured at about 3400 centipoise using a Brookfield DV-1 viscometer with an LV-4 spindle set at 100 rpm. The calculated concentration of each component of the aqueous formulation is set forth below in Table 3.

TABLE 3
Components of the Aqueous Formulation

| Component | Calculated Amount |
|---|---|
| Magnesium Chloride | 33.3% |
| Xanthan gum | 1.1% |
| Water | 65.6% |

The aqueous formulation was applied to the same dual layer bonded carded web as described in Example 1. The coated fabric pieces were placed in a laboratory oven at 110° C. for about 30 minutes to dry. The concentration of the components of the coating composition was calculated from the coated and dried fabric pieces (23.6±1.4 grams), the untreated pieces of fabric (2.92±0.01 grams), and the composition of the aqueous formulation. The results are set forth below in Table 4.

TABLE 4
Components of the Coating Composition

| Components | Calculated Amount |
|---|---|
| Magnesium Chloride | 96.8% |
| Xanthan Gum | 3.2% |
| Solids Add-On Level | ~708% |

A sleeve structure (3″×10.5″) was then designed for activating the warming reaction. Specifically, the sleeve structure was produced with a material obtained from Shanghai Intco Medical Supply Co., Ltd./Shanghai Intco Electrode Manufacturing Co., Ltd. (No. 1299, Hubin Rd, Fengxian district, Shanghai China). The material contained an 8 mil thermoplastic polyurethane film laminated to a spunlace nonwoven fabric. The sleeve was constructed with the film layer on the inside and the fabric layer on the outside. The total basis weight of this film/fabric laminate was measured at 174 grams per square meter and the thickness was measured at 0.60 mm (0.023 in) using a Mitutoyo Digimatic Indicator. Four pieces (2.2″ CD×7″ MD) of the coated fabric (total weight of 26.8 grams) were placed inside of the sleeve. Two pieces were placed together with the coated side of each piece aligned to face one side of the sleeve. The other two pieces were also placed together with the coated side of each piece aligned to face the opposite side of the sleeve. Two water bags were also placed inside of the film sleeve. The water bags were made out of GF-14 film (a 1.25 mil low density polyethylene barrier film from Pliant Corp.) and filled with 17.7 and 17.8 grams of water. The bags were constructed by folding over a 3.5-inch by 2.5-inch piece of the film and heat sealing two edges. The water was then added with a plastic syringe and the third edge was then heat sealed. The final dimensions of the bags were about 1.5 inches by 2.5 inches. A water bag was positioned at each end of the sleeve. The film/fabric laminate that formed the sleeve was also heat sealed to enclose the coated fabric pieces and the water bags. The sleeve was evacuated prior to the final heat seal by sealing with a V-300 Fuji Impulse Vacuum Sealer.

Data from the thermocouples were collected as described in Example, 1 and are represented in FIG. 8B. An additional temperature probe was used to monitor ambient background temperature. Note that this particular formulation produced an almost immediate temperature increase of about 10 degrees (° C.), and maintained an increased temperature for over 30 minutes.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A warming device, comprising:
a shell member having an outer face and an inner face disposed against a surface to be warmed in use of said device, said shell member defining an interior space;
a first warming substrate disposed within said interior space, said warming substrate comprising an absorbent web having a generally uniform application of a first warming composition applied thereto, said warming composition activated by contact with an aqueous liquid;
an aqueous liquid source disposed within said interior space and separated from said first warming substrate by a barrier member; and
said device activated by manual manipulation to breach said barrier member causing liquid from said liquid source to move within said interior space to contact and activate said warming composition whereby a warming reaction is generated.

2. The warming device as in claim 1, wherein said shell member has sufficient rigidity to maintain its configuration when said device is not in use.

3. The warming device as in claim 1, wherein said shell member comprises a flexible and conformable sleeve member that conforms around an object to be warmed, said first warming substrate comprising a flexible material so as to conform with said shell member around the object.

4. The warming device as in claim 3, wherein said shell member comprises opposite ends, and further comprising an attaching mechanism configured with said ends, wherein said sleeve member is wrapped around and attached to the object to be warmed.

5. The warming device as in claim 1, wherein said first warming composition has a first set of warming characteristics, and further comprising a second warming composition within said interior space having a different second set of warming characteristics.

6. The warming device as in claim 5, wherein said second warming composition is applied to said first warming substrate.

7. The warming device as in claim 1, further comprising a second warming substrate disposed within said interior space, said second warming composition applied to said second warming substrate.

8. The warming device as in claim 1, wherein said absorbent web comprises a porous material, said first warming composition is an aqueous solution coated onto said porous material so as to form a film-like network distributed throughout said porous material when dried, said warming composition further comprising at least one of a binder or viscosity modifier.

9. The warming device as in claim 8, wherein said warming agent comprises an inorganic salt.

10. The warming device as in claim 9, wherein said warming composition comprises a polysaccharide.

11. The warming device as in claim 10, wherein said warming substrate comprises at least 25% by weight of said inorganic salt, and at least 0.5% by weight of said polysaccharide.

12. The warming device as in claim 10, wherein said warming substrate comprises at least 50% by weight of said inorganic salt, and at least 1% by weight of said polysaccharide.

11. The warming device as in claim 1, wherein said liquid source comprises liquid stored in a compartment defined within said interior space and formed at least in part by said interior surfaces of said shell member, said barrier member comprising a wall of said compartment that separates or breaks upon manual manipulation of said device to release liquid contained in said compartment.

12. The warming device as in claim 1, wherein said liquid source comprises at least one bladder inserted into said interior space, said bladder separating or breakable upon manual manipulation of said device.

13. The warming device as in claim 1, further comprising an insulation material layer adjacent said outer face of said shell member.

14. The warming device as in claim 1, further comprising a thermal conductive material layer within said interior space disposed so as to direct warmth generated in the reaction towards said inner face of said shell member.

15. The warming device as in claim 1, wherein substantially all of said liquid from said liquid source is absorbed by said warming substrate so that excess liquid is not held within said interior space after activation of said device.

16. The warming device as in claim 1, wherein said warming substrate comprises a separate material disposed within said interior space between interior faces of said shell member.

17. The warming device as in claim 1, wherein said warming substrate comprises an interior material layer of said shell member.

18. The warming device as in claim 1, further comprising a plurality of said warming substrates within said interior space.

19. The warming device as in claim 18, wherein each of said warming substrates generates different warming characteristics upon activation of said device.

20. The warming device as in claim 19, wherein each of said warming substrates comprises a different warming composition.

21. The warming device as in claim 18, further comprising a material separator layer between adjacent said warming substrates.
